(19) United States
(12) Patent Application Publication
(10) Pub. No.: US 2006/0135774 A1
(43) Pub. Date: Jun. 22, 2006

(54) Method for producing pigment transparent preparations based on perylene-3,4,9,10-tetracarboxylic acid diimine

(76) Inventors: Joachim Weber, Reinach (CH); Manfred Opravil, Karben (DE); Erwin Dietz, Koenigstein (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/546,500

(22) PCT Filed: Jan. 31, 2004
(86) PCT No.: PCT/EP04/00868

§ 371 Date: Aug. 19, 2005

(30) Foreign Application Priority Data
Feb. 21, 2003 (DE) 103 07 557.7

(51) Int. Cl. C07D 221/22; C07D 471/02

(52) U.S. Cl. 546/37

(57) ABSTRACT

The invention relates to a method for producing pigment transparent preparations based on perylene-3,4,9,10-tetracarboxylic acid diimine of (XXX) formula, wherein u is a number between 0 and 8, when u>0; E is a chlorine or bromine atom and if u>1, possibly the combination of said atoms. The inventive method consists in wet grinding a perylimide raw pigment of the (xxx) formula in an aqueous liquid medium in the presence of at least one pigment dispersing agent of a perylene dispersing agent group and P.V. derived dispersing agents with the aid of a ball grinding mill operating at a maximum volume power of 1.0 kW per litre by the action of a grinder whose diameter is equal to or less than 5 mm, at a temperature equal to or higher than 50° C., and in isolating the such obtained pigment preparation.

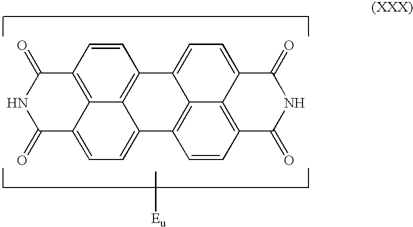

Process for preparing transparent pigment preparations based on perylene-3,4,9,10-tetracarboxylic diimide

The present invention relates to a particularly environment-friendly and economic process for preparing transparent pigment preparations based on perylene-3,4,9,1 0-tetracarboxylic diimide

In the pigmentation of automotive finishes, especially the metallic finishes, there is a need for pigments which with a high transparency and clean hue give strongly colored and highly glossy finishes. The pigment concentrates and the paints must be of low viscosity and must not exhibit pseudoplasticity. Moreover, very good fastness properties are demanded, such as weatherfastness and fastness to overcoating, for example. The pigments should be able to be used in both solventborne and aqueous paint systems.

Depending on the synthesis route, organic pigments are obtained in coarsely crystalline or finely divided form. Crude pigments obtained in coarsely crystalline form require fine division before being used. Examples of such fine division processes are grinding on roll mills or vibratory mills, which may be performed wet or dry, with or without grinding aids such as salt, for example; kneading, revatting, reprecipitation from sulfuric acid or polyphosphoric acid, for example (acid pasting), and suspending in sulfuric acid or polyphosphoric acid, for example (acid swelling).

In the case of perylene-3,4,9,10-tetracarboxylic diimide (referred to below as perylimide), neither the prepigments obtained from a synthesis nor the prepigments obtained in finely divided form from a fine division known to date may be used directly as pigments, since in the course of processing and especially in the course of drying they have a very strong tendency to form aggregates and agglomerates, leading to poor dispersibility, which results, for example, in a loss of color strength. Moreover, perylimide pigments exhibit a very strong tendency to flocculate in paints. Both phenomena result in performance properties which no longer satisfy the present-day requirements.

Perylimide has been used for a long time as an organic brown to reddish violet pigment (C.I. Pigment Violet 29, C.I. No. 71129). The suitability of the halogenated derivatives as pigments is also known. The crude pigment is prepared either by reacting 1,8-naphthalenedicarboximide in alkali metal hydroxide melts and then oxidizing the resultant leuco form, as described for example in EP-A-0 123 256, or by condensing perylene-3,4,9,10-tetracarboxylic acid or its anhydrides or carboxylic salts (called peracid below) with ammonia, as described for example in DE-A-386 057. The preparation of the halogenated derivatives is described, for example, in EP-A-0 260 648. The following processes are described for converting the resultant perylimides into a coloristically useful pigment form:

DE-A-20 43 820 describes a wet grinding process in an aqueous-alkaline medium. The pigments obtained under the stated conditions, however, no longer satisfy present-day requirements.

EP-A-1 130 062 describes the conversion of crude perylimide into a pigment by wet grinding with specific beadmills which are characterized by a high energy input. These mills are expensive both to acquire and to operate.

There was a need to prepare transparent perylimide pigments in a simple, cost-effective and environmentally unalloyed manner in a one-stage process.

It has been found that transparent perylimide pigment preparations having advantageous coloristic and rheological properties may be prepared in a simple and environmentally unalloyed way from crude perylimide pigments by aqueous wet grinding in a low-energy, stirred ballmill in the presence of at least one pigment dispersant from the group of perylene dispersants and the dispersants derived from P.V.23, at alkaline pH and at elevated temperature.

The present invention provides a process for preparing transparent perylimide pigment preparations based on perylene-3,4,9,10-tetracarboxylic diimides of the formula (XXX) 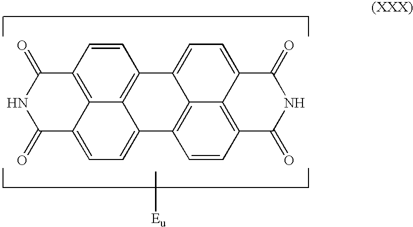
in which
u is a number from 0 to 8 and, if u>0,
E is a chlorine or bromine atom and, where u>1, may be a combination thereof, characterized in that a crude perylimide pigment of the formula (XXX) is wet-ground in a liquid, aqueous medium, in the presence of at least one pigment dispersant from the group of perylene dispersants and dispersants derived from P.V.23, in a stirred ballmill operated with a power density of not more than 1.0 kW per liter of milling space, under the action of grinding media having a diameter of less than or equal to 5 mm and at a temperature of at least 50° C., and isolating the resulting pigment preparation.

The pigment preparations of the invention may also comprise mixtures of two or more perylimide pigments of the formula (XXX) as base pigment.

The crude perylimide pigment may have been prepared either by reaction of 1,8-naphthalenedicarboximide in alkali metal hydroxide melts with subsequent oxidation of the leuco form or by condensation from peracid, as defined above, with ammonia.

The crude, coarsely crystalline perylimide pigments obtainable from the synthesis, or finely crystalline perylimide prepigments, may be supplied in powder form or advantageously as a suspension, or in the form of an as-synthesized moist presscake, without further drying, to the wet grinding operation. It is also possible to purify the crude perylimide pigments, for example, by recrystallization or by extractive stirring with, for example, sulfuric acid.

The pigment dispersants used in the process of the invention and derived from P.V.23 are known per se. Preference is given to compounds of the formula (I) 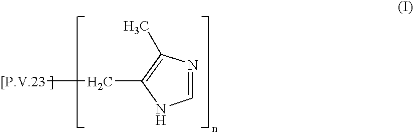
in which
n is a number from 1 to 4, preferably 1 to 2.

The perylene dispersants used in the process of the invention are known per se. Preference is given to compounds of the formula (II) 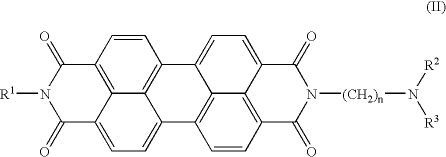
in which
R is a hydrogen atom, a hydroxyl or amino group or a C₁-C₈-alkyl group which may be substituted by from 1 to 4 chlorine or bromine atoms or by a phenyl, cyano, hydroxyl, carbamoyl, C₂-C₄-acyl or C₁-C₄-alkoxy group or is perfluorinated or partly fluorinated;
R² and R³ independently of one another are a hydrogen atom, a substituted or unsubstituted or partly fluorinated or perfluorinated alkyl group of 1 to 20 carbon atoms, or a substituted or unsubstituted or partly fluorinated or perfluorinated alkenyl group of 2 to 20 carbon atoms, it being possible for the substituents to be hydroxyl, phenyl, cyano, chlorine, bromine, C₂-C₄-acyl or C₁-C₄-alkoxy;
5- to 7-membered or R² and R³ together with the nitrogen atom form a saturated, unsaturated or aromatic heterocyclic ring which if desired contains a further nitrogen, oxygen or sulfur atom in the ring; and
n is a number from 1 to 6;
or of the formula (III) 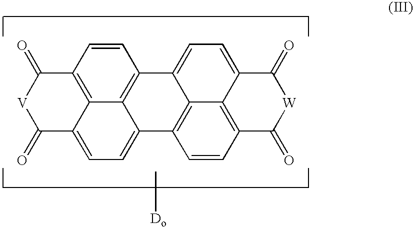
in which
V is a bivalent radical —O—, >NR or >NR⁵—Y⁻X⁺,
W is the bivalent radical >NR⁵—Y⁻X⁺,
o is a number from 0 to 8, preferably from 0 to 4, and, if o>0,
D is a chlorine or bromine atom and, where o>1, may be a combination thereof,
R is a hydrogen atom or a C₁-C₁₈-alkyl group, especially C₁-C₄-alkyl, or is a phenyl group which may be unsubstituted or substituted one or more times, preferably 1, 2 or 3 times, by halogen, such as chlorine or bromine, C₁-C₄-alkyl, such as methyl or ethyl, C₁-C₄-alkoxy, such as methoxy or ethoxy, or phenylazo,
R is a C₁-C₁₈-alkylene group which within the C—C chain may be interrupted one or more times by a bridging link from the series —O—, —NR⁵—, —S—, phenylene, —CO—, —SO₂— or —CR⁷R⁸ — or by a chemically rational combination thereof and in which the radicals R⁶, R⁷ and R⁸ independently of one another are each a hydrogen atom or a C₁-C₄-alkyl group which may be unsubstituted or substituted by a heterocyclic radical, preferably of the imidazole or piperazine type, and in particular is a straight-chain or branched C₁-C₆-alkylene group such as ethylene or propylene;
   or R⁵ is a phenylene group which may be unsubstituted or substituted one or more times, preferably 1, 2 or 3 times, by C₁-C₄-alkyl, such as methyl or ethyl, or C₁-C₄-alkoxy, such as methoxy or ethoxy,
Y⁻ is one of the anionic radicals —SO₃⁻ or —COO⁻, and
X⁺ has the definition H⁺ or the equivalent

$$\frac{{M}^{m +}}{m}$$

of a metal cation from main groups 1 to 5 or transition groups 1 or 2 or 4 to 8 of the Periodic Table of the Chemical Elements, m being one of the numbers 1, 2 or 3, and M^{m+} being, for example, Li¹⁺, Na¹⁺, K¹⁺, Mg²⁺, Ca²⁺, Sr²⁺, Ba²⁺, Mn²⁺, Cu²⁺, Ni²⁺, Co²⁺, Zn²⁺, Fe²⁺, Al³⁺, Cr³⁺ or Fe³⁺;
or defines an ammonium ion N⁺R⁹R¹⁰R¹¹R¹², where the substituents R⁹, R¹⁰, R¹¹ and R¹² independently of one another are each a hydrogen atom or a group from the series C₁-C₃₀-alkyl, C₂-C₃₀-alkenyl, C₅-C₃₀-cycloalkyl, unsubstituted or C₁-C₈-alkylated phenyl, (C₁-C₄)-alkylene-phenyl, preferably benzyl, or a (poly)alkylenoxy group 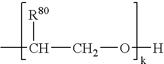
in which k is a number from 1 to 30,
R⁸⁰ is hydrogen, C₁-C₄-alkyl or, if k is >1, a combination thereof;
and in which R⁹, R¹⁰, R¹¹ and R¹² identified as alkyl, alkenyl, cycloalkyl, phenyl or alkylphenyl may be substituted by amino, hydroxyl and/or carboxyl;
or where the substituents R⁹ and R¹⁰ together with the quaternary nitrogen atom may form a five- to seven-membered saturated ring system which if desired contains further heteroatoms from the group O, S and N, e.g., a pyrrolidone, imidazolidine, hexamethyleneimine, piperidine, piperazine or morpholine;
or where the substituents R⁹, R¹⁰ and R¹¹ together with the quaternary nitrogen atom may form a five- to seven-membered aromatic ring system which if desired contains further heteroatoms from the group O, S and N and to which, if desired, additional rings are fused on, e.g., a pyrrole, imidazole, pyridine, picoline, pyrazine, quinoline or isoquinoline;
or of the general formula (IV) 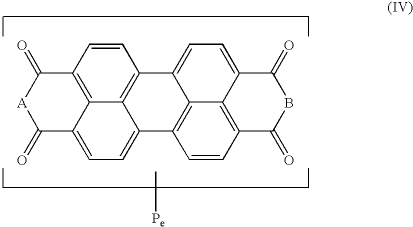
in which
A is a cationic bivalent radical of the formula N—R¹³—NHR¹⁴R¹⁵,
B is an anionic bivalent radical of the formula 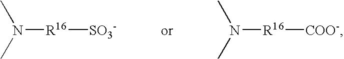
e is a number from 0 to 8, preferably 1 to 6, and, if e is >0,
P is a chlorine or bromine atom and, where e is >1, may be a combination thereof,
R¹³ is a C₁-C₁₂-alkenylene group, preferably a C₂-C₆-alkenylene group, a (C₆-C₁₀)-aryl-(C₁-C₆)-alkylene group or a (C₆-C₁₀)-arylene group, preferably phenylene,
R¹⁴ and R¹⁵ are identical or different and are a hydrogen atom, C₁-C₂₀-alkyl, preferably C₁-C₆-alkyl, or C₂-C₂₀-alkenyl, or
R¹⁴ and R¹⁵ together with the adjacent nitrogen atom form a heterocyclic ring system which if desired contains further heteroatom ring members N, S and/or O and to which, if desired, additional rings are fused on, and
R¹⁶ is a straight-chain or branched C₁-C₁₂-alkylene group, preferably C₁-C₆-alkylene group;
or of the general formula (V) 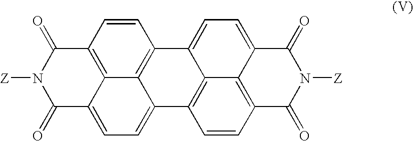
   in which the two radicals Z are identical or different and Z has the definition Z¹, Z³ or Z⁴, with the proviso that both radicals Z are not simultaneously Z⁴, where
Z is a radical of the formula (Va)
   —[X—Y]_{q}—[X¹—Y¹]ᵣ—[X²—NH]ₛH (Va)
in which
X, X¹ and X² are identical or different and are a branched or unbranched C₂-C₆-alkylene radical or a C₅-C₇-cycloalkylene radical which may be substituted by from 1 to 4 C₁-C₄-alkyl radicals, hydroxyl radicals, hydroxyalkyl radicals of 1 to 4 carbon atoms, and/or by 1 or 2 further C₅-C₇-cycloalkyl radicals;
Y and Y¹ are identical or different and are an NH, —O—, N(C₁-C₆-alkyl) group, preferably —NCH₃, or 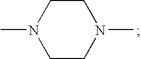
q is a number from 1 to 6, preferably 1, 2, 3 or 4;
r and s independently of one another are a number from 0 to 6, preferably 0, 1 or 2, r and s preferably not simultaneously being zero;
Z³ is a radical of the formula (Vc) 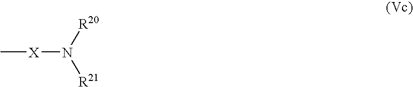
in which
R²⁰ and R²¹ independently of one another are a hydrogen atom, a substituted or unsubstituted, or partly fluorinated or perfluorinated alkyl group of 1 to 20 carbon atoms, or a substituted or unsubstituted, or partly fluorinated or perfluorinated alkenyl group of 2 to 20 carbon atoms, it being possible for the substituents to be hydroxyl, phenyl, cyano, chlorine, bromine, C₂-C₄-acyl or C₁-C₄-alkoxy, or
R²⁰ and R²¹ together with the nitrogen atom form a saturated, unsaturated or aromatic heterocyclic 5- to 7-membered ring which if desired contains a further nitrogen, oxygen or sulfur atom in the ring; and
x is as defined above;
and in which
Z⁴ is hydrogen, hydroxyl, amino, phenyl or C₁-C₂₀-alkyl, it being possible for the phenyl ring and the alkyl group to be substituted by one or more, preferably 1, 2, 3 or 4, substituents from the group consisting of Cl, Br, CN, OH, C₆H₅, carbamoyl, C₂-C₄-acyl and C₁-C₄-alkoxy, and the phenyl ring may also be substituted by NR²⁰R²¹, R²⁰ and R²¹ being as defined above, or the alkyl group is perfluorinated or partly fluorinated.

Depending on the choice of radicals Z, symmetric or asymmetric perylene dispersants can be employed, asymmetric perylene dispersants also including those having different radicals Z¹ or Z³.

Perylene dispersants of interest are, for example, those of formula (V) in which X, X¹ and X² are a C₂-C₄-alkylene radical or cyclohexylene.

Of particular interest are, for example, perylene dispersants of formula (V) in which Z has one of the following definitions: —[(CH₂)₃—NH]₂—H, —(CH₂—CH₂—NH)₂H, —(CH₂)₃—NH—(CH₂)₂—NH—(CH₂)₃—NH₂, 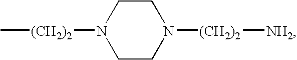
—(CH₂)₃—N(CH₃)—(CH₂)₃—NH₂, —(CH₂)₃—O—(CH₂)₂—O—(CH₂)₃—NH₂, —(CH₂)₃—O—(CH₂)₃—O—(CH₂)₃—NH₂, —(CH₂)₂—NH—(CH₂)₃—NH₂, —(CH₂)₃—NH—(CH₂)₂—NH₂,
—(CH₂—CH₂—NH)₃—H, —(CH₂—CH₂—NH)₄—H, —(CH₂—CH₂—NH)₅—H, —(CH₂)₃—O—(CH₂)₂—O—(CH₂)₂—O—(CH₂)₃—NH₂, —(CH₂)₃—O—(CH₂)₄—O—(CH₂)₃—NH₂, 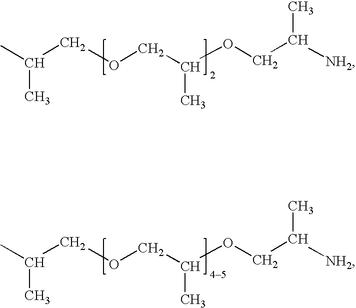
   —(CH₂)₂—OH, —(CH₂)₃—OH, —CH₂—CH(CH₃)—OH, —CH(CH₂—CH₃)CH₂—OH, —CH(CH₂OH)₂, —(CH₂)₂—O—(CH₂)₂—OH, —(CH₂)₃—O—(CH₂)₂O—(CH₂)₂—OH.

Preferably the radicals R²⁰ and R²¹ are hydrogen, C₁-C₆-alkyl, C₁-C₆-alkyl substituted by 1 or 2 substituents from the group consisting of hydroxyl, acetyl, methoxy, ethoxy, chlorine and bromine; or R and R together with the adjacent nitrogen atom form an imidazolyl, piperidinyl, morpholinyl, pipecolinyl, pyrrolyl, pyrrolidinyl, pyrazolyl or piperazinyl ring.

Preferably Z is —(CH₂)₂—NH₂, —(CH₂)₃—NH₂, —CH₂—CH(CH₃)—NH₂, 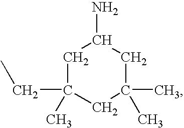
—H₂—C(CH₃)₂—CH₂—NH₂, 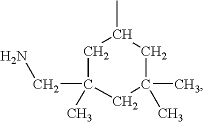
   —(CH₂)₂—NH—CH₃, —(CH₂)₂—N(CH₃)₂, —(CH₂)₂—NH—CH₂—CH₃, —(CH₂)₂—N(CH₂—CH₃)₂, —(CH₂)₃—NH—CH₃, —(CH₂)₃—N(CH₃)₂, —(CH₂)₃—NH—CH₃ or
—(CH₂)₃—N(CH₂—CH₃)₂—.

Preferably Z⁴ is hydrogen, amino, phenyl, benzyl, NR²⁰R²¹-substituted phenyl or benzyl, C₁-C₆-alkyl or a C₂-C₆-alkyl substituted by 1 to 2 substituents from the group consisting of hydroxyl, acetyl, methoxy and ethoxy, with particular preference hydrogen, 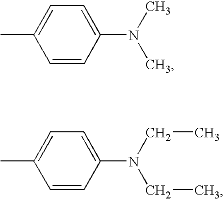
methyl, ethyl, propyl, butyl, benzyl, hydroxyethyl, hydroxypropyl or methoxypropyl.

Particularly preferred pigment dispersants are those of the formula (II) in which
R¹ is a hydrogen atom, benzyl, a C₁-C₆-alkyl group or a C₂-C₆-alkyl group substituted by 1 or 2 substituents from the group consisting of hydroxyl, acetyl, methoxy, ethoxy, chlorine and bromine;
R² and R³ independently of one another are a hydrogen atom, a C₁-C₆-alkyl group, a C₁-C₆-alkyl group substituted by 1 or 2 substituents from the group consisting of hydroxyl, acetyl, methoxy, ethoxy, chlorine and bromine, or R² and R³ together with the adjacent nitrogen atom form an imidazolyl, piperidinyl, morpholinyl, pipecolinyl, pyrrolyl, pyrrolidinyl, pyrazolyl or piperazinyl ring, and
n is the number 2 or 3.

Of particular interest are pigment dispersants of the formula (II) in which R¹ is hydrogen, hydroxyethylene, methyl or ethyl, R² and R³ are each methyl or ethyl and n is the number 3, or in which R² and R³ together with the adjacent nitrogen atom form an imidazolyl radical or morpholinyl radical and n is the number 3.

Particular preference is further given to pigment dispersants of the formula (IV) in which
e is the number 0,
R¹³ is an ethylene or propylene group,
R¹⁴ and R¹⁵ are different or, preferably, identical and are an ethyl, propyl or butyl radical, or
R¹⁴ and R¹⁵ together with the adjacent nitrogen atom form a heterocyclic aliphatic five- or six-membered ring with 1 or 2 further heteroatom ring members N, S and/or O, such as, for example, of the pyrrolidine, piperazine and, in particular, piperidine or morpholine type, or
R¹⁴ and R¹⁵ analogously, together with the adjacent nitrogen atom, form a heterocyclic aromatic five- or six-membered ring, such as, for example, of the pyrrole, imidazoline and, in particular, imidazole type,
   it being possible for the ring also to have a benzo-fused ring, such as, for example, of the indole, indoline or benzimidazole type, and for the ring system to be substituted by methyl, ethyl or chlorine, and
R¹⁶ is an ethylene or propylene group.

The total amount of pigment dispersants used is 0.1% to 25%, preferably 0.5% to 20%, in particular 1% to 15% by weight, based on the crude pigment.

The preparation of transparent perylimide pigment preparations in accordance with the invention is surprisingly achieved without dry grinding beforehand. Mills suitable for conducting the wet grinding of the invention are conventional stirred ballmills which are designed for batchwise and continuous operation, which have a cylindrical or hollow-cylindrical milling chamber in horizontal or vertical construction, and which can be operated with a specific power density of not more than 1.0 kW per liter of milling space, preferably between 0.1 and 1.0 kW per liter of milling space, their peripheral stirrer speed advantageously being not more than 12 m/s, preferably 2 to 12 m/s, in particular 5 to 11 m/s. The constructional design ensures that a sufficient milling energy is transferred to the millbase. The energy output per unit time by the stirrer mechanism is transmitted to the millbase as disruption energy and as frictional energy in the form of heat. In order to remove the quantity of heat, it is possible to cool the mill. At high throughputs, milling is carried out in circulation and the heat can be dissipated to the outside predominantly via the millbase. Grinding media used are beads made, for example, of steel, porcelain, steatite, oxides such as aluminum oxide or zirconium oxide, for example, mixed oxides such as zirconium mixed oxide, for example, or of glass, such as quartz glass, for example, with a diameter of less than or equal to 5 mm; it is advantageous to use those having a diameter of 0.2 to 5 mm, preferably from 0.3 to 3 mm, in particular 0.5 to 2 mm or 1.0 to 2 mm.

When continuous stirred ballmills are used for the fine division, the grinding media are separated from the millbase preferably by centrifugation, so that there is virtually no contact between the separation devices and the grinding media, thereby making it possible to a large extent to prevent the separation devices becoming blocked.

Grinding is carried out in a liquid, aqueous medium, with the addition where appropriate of small amounts of up to 10% by weight of organic solvents, based on the water.

Solvents suitable as the organic solvent added, if desired, in small amounts include C₁-C₈-alkanols, advantageously water-miscible alkanols, such as methanol, ethanol, n-propanol, isopropanol, n-butanol, tert-butanol, isobutanol, pentanol, hexanol, alkylhexanols, ethylene glycol, propylene glycol or glycerol, for example; cyclic alkanols such as cyclohexanol, for example; C₁-C₅ dialkyl ketones such as acetone, diethyl ketone, methyl isobutyl ketone or methyl ethyl ketone, for example; ethers and glycol ethers such as dimethoxyethane, tetrahydrofuran, methyl glycol, ethyl glycol, butyl glycol, ethyl diglycol, methoxypropanol or methoxybutanol, for example; aliphatic acid amides such as dimethylacetamide or dimethylformamide, for example; cyclic carboxamides such as N-methylpyrrolidone, valerolactam and caprolactam, for example, heterocyclic bases such as pyridine, morpholine or picoline, for example; and also dimethyl sulfoxide, or mixtures of these solvents. Preference is given to C₁-C₆ alcohols, but with particular preference operation takes place without addition of organic solvent.

The pigment concentration in the millbase is dependent on the rheology of the suspension. It should be below 40% by weight, generally from 2.5% to 30% by weight, preferably between 5% and 20% by weight. An advantageous pH is a pH greater than or equal to 9.0, preferably greater than or equal to 10. It is usual to operate at a pH greater than or equal to 11.0.

The milling duration is dependent on the fineness requirements for the field of use in question. Accordingly, depending on the required fineness, the residence time of the millbase in the stirred ballmill is generally between 10 minutes and 5 hours. The residence time normally runs to a duration of 15 minutes to 2 hours, preferably 15 minutes to 1 hour.

Milling is conducted at temperatures in the range from 50 to 100° C., advantageously at a temperature between 60 and 95° C., preferably at 70 to 90° C.

Besides the liquid phase and the crude pigment, the millbase may further comprise auxiliaries, such as surfactants, other pigment dispersants, fillers, standardizers, resins, defoamers, antidust agents, extenders, shading colorants, preservatives, drying retardants, rheology control additives, or a combination thereof, for example.

The addition of the aforementioned auxiliaries may take place at one or more arbitrary points in time in the course of the overall pigment preparation process, preferably prior to condensation or prior to wet grinding, but also during condensation, during wet grinding, before or during isolation, before or during drying, or to the dry pigment (powder or granules), all at once or in two or more portions. The overall amount of the auxiliaries added may be from 0 to 40% by weight, preferably from 1 to 25% by weight, with particular preference from 5 to 15% by weight, based on the crude pigment.

Suitable surfactants include anionic or anion-active, cationic or cation-active, and nonionic substances, or mixtures of these agents. Preference is given to those surfactants or surfactant mixtures which do not foam during the distillation of the ammonia and in the course of the wet grinding.

Examples of suitable anion-active substances are fatty acid taurides, fatty acid N-methyl taurides, fatty acid isethionates, alkylphenylsulfonates, alkylnaphthalenesulfonates, alkylphenol polyglycol ether sulfates, fatty alcohol polyglycol ether sulfates, fatty acid amide polyglycol ether sulfates, alkylsulfosuccinamates, alkenylsuccinic monoesters, fatty alcohol polyglycol ether sulfosuccinates, alkanesulfonates, fatty acid glutamates, alkylsulfosuccinates, fatty acid sarcosides; fatty acids, e.g., palmitic, stearic and oleic acid; soaps, e.g., alkali metal salts of fatty acids, naphthenic acids and resin acids, e.g., abietic acid, alkali-soluble resins, e.g., rosin-modified maleate resins and-condensation products based on cyanuric chloride, taurine, N,N′-diethylaminopropylamine and p-phenylenediamine. Particular preference is given to resin soaps, i.e., alkali metal salts of resin acids.

Examples of suitable cationic substances are quaternary ammonium salts, fatty amine alkoxylates, alkoxylated polyamines, fatty amino polyglycol ethers, fatty amines, diamines and polyamines derived from fatty amines or fatty alcohols, and their alkoxylates, imidazolines derived from fatty acids, and salts of these cationic substances.

Examples of suitable nonionic substances are amine oxides, fatty alcohol polyglycol ethers, fatty acid polyglycol esters, betaines, such as fatty acid amide-N-propyl betaines, phosphoric esters of fatty alcohols or fatty alcohol polyglycol ethers, fatty acid amide ethoxylates, fatty alcohol-alkylene oxide adducts, and alkylphenol polyglycol ethers.

By fillers and/or extenders are meant a multiplicity of substances in accordance with DIN 55943 and DIN EN 971-1, examples being the various types of talc, kaolin, mica, dolomite, lime, barium sulfate or titanium dioxide.

The pigment preparation is preferably isolated directly after wet grinding. It is possible, however, to carry out an aftertreatment (finish) with water and/or an organic solvent as described above, at temperatures of 20 to 180° C., for example.

It is also possible to concentrate the as-ground pigment suspension or to subject it to spray drying, so that filtration can be omitted.

The preparation of the perylimide pigment preparations by the process of the invention takes place essentially without waste products. The few chemicals there are can be processed further or fully regenerated.

It was surprising and unforeseeable that the preparation of transparent perylimide pigment preparations would be possible in this simple and technically elegant way without environmental problems, since in accordance with the known processes, particularly the teaching of EP-A-1 130 062, the preparation of transparent perylimide pigments is accomplished only through the use of high-energy stirred ballmills which are expensive to acquire and operate. In terms of their coloristic and performance properties, perylimide pigment preparations prepared by the process of the invention are superior to the pigments prepared by known processes.

Using the perylimide pigment preparations prepared by the process of the invention it is possible to produce automotive finishes, especially metallic finishes with high pigment concentration. They are suitable for use both in solventborne and in aqueous paint systems. Transparent and glossy finishes of high color strength are obtained with very good fastness to overcoating and weather fastness. The pigment concentrates (millbases) and the paints further possess very good flow properties, with a high pigment concentration and outstanding flocculation stability.

Perylimide pigment preparations prepared in accordance with the invention are suitable for pigmenting high molecular mass natural or synthetic organic materials, such as cellulose ethers and cellulose esters, such as ethylcellulose, nitrocellulose, cellulose acetate, cellulose butyrate, natural resins or synthetic resins, such as addition polymerization resins or condensation resins, e.g., amino resins, especially urea-formaldehyde and melamine-formaldehyde resins, alkyd resins, acrylic resins, phenolic resins, polycarbonates, polyolefins, such as polystyrene, polyvinyl chloride, polyethylene, polypropylene, polyacrylonitrile, polyacrylates, polyamides, polyurethanes or polyesters, rubber, casein, silicone and silicone resins, individually or in mixtures.

In this context it is unimportant whether the abovementioned high molecular mass organic compounds are present in the form of plastic masses, melts, spinning solutions, varnishes, paints or printing inks. Depending on the intended use it is found advantageous to utilize the pigments obtained in accordance with the invention as blends or in the form of prepared formulations or dispersions. Based on the high molecular mass organic material to be pigmented, the pigment preparations prepared in accordance with the invention are used in an amount of preferably from 0.05 to 30% by weight, more preferably from 0.1 to 15% by weight.

Using the pigment preparations prepared by the process of the invention it is possible to pigment the stoving enamels customary in the art from the class of the alkyd melamine resin varnishes, acrylic melamine varnishes, polyester varnishes, high solid acrylic resin varnishes, aqueous varnishes based on polyurethane, and also two-component varnishes based on polyisocyanate crosslinkable acrylic resins, and especially automotive metallic varnishes.

The pigment preparations prepared in accordance with the invention are also suitable as colorants in electrophotographic toners and developers, such as one- or two-component powder toners (also called one- or two-component developers), magnetic toners, liquid toners, polymerization toners, and specialty toners.

Typical toner binders are addition polymerization, polyaddition and polycondensation resins, such as styrene, styrene acrylate, styrene butadiene, acrylate, polyester, phenolepoxy resins, polysulfones, polyurethanes, individually or in combination, and also polyethylene and polypropylene, which may include further ingredients, such as charge control agents, waxes or flow assistants, or may be modified subsequently with these additives.

Furthermore, the pigment preparations prepared in accordance with the invention are suitable as colorants in powders and powder coating materials, especially in triboelectrically or electrokinetically sprayable powder coating materials, which are used to coat the surfaces of articles made, for example, of metal, wood, plastic, glass, ceramic, concrete, textile material, paper or rubber.

Typical powder coating resins used comprise epoxy resins, carboxyl- and hydroxyl-containing polyester resins, polyurethane resins and acrylic resins, together with customary hardeners. Resin combinations are also used. For example, epoxy resins are frequently used in combination with carboxyl- and hydroxyl-containing polyester resins. Typical hardener components (depending on the resin system) are, for example, acid anhydrides, imidazoles and also dicyandiamide and its derivatives, blocked isocyanates, bisacylurethanes, phenolic resins and melamine resins, triglycidyl isocyanurates, oxazolines, and dicarboxylic acids.

In addition, the pigment preparations prepared in accordance with the invention are suitable as colorants in inks, preferably ink-jet inks on either an aqueous or nonaqueous basis, in microemulsion inks and also in those inks which operate in accordance with the hot-melt technique.

Furthermore, the pigment preparations prepared in accordance with the invention are also suitable as colorants for color filters, both for subtractive and for additive color generation, and also as colorants for electronic inks (or “e-inks”) or electronic paper (“e-paper”).

In order to evaluate the properties in the coatings sector of the pigment preparations prepared in accordance with the invention, a selection was made from among the large number of known varnishes of a high-solid acrylic resin baking varnish based on a nonaqueous dispersion (HS).

The color strength and shade were determined in accordance with DIN 55986.

The rheology of the millbase after dispersion (millbase rheology) was evaluated on the basis of the following five-point scale:
5 highly fluid
4 liquid
3 viscous
2 slightly set
1 set

Following dilution of the millbase to the final pigment concentration, the viscosity was evaluated using the Rossmann viscospatula, type 301 from Erichsen.

Gloss measurements were carried out on cast films at an angle of 20° in accordance with DIN 67530 (ASTMD 523) using the “multigloss” gloss meter from Byk-Mallinckrodt.

In the examples below, parts and percentages are based in each case on the weight of the substances so described.

### EXAMPLE 1

1100 parts of glass beads with a diameter of 1 mm as grinding media, 300 parts of water and 0.6 part of sodium hydroxide, which sets a pH of 12.3, are heated to 80° C. in a 1 liter milling pot of a stirred ballmill. Then 32 parts of crude perylimide, prepared from the alkali metal hydroxide melt of 1,8-naphthalenedicarboximide, and 8.2 parts of 29.1% presscake of pigment dispersant (P.V.23-5-hydroxymethyl-4-methylimidazolyl), prepared according to EP-A-0 321 919 example 17, are introduced and the millbase is milled for 30 minutes at a peripheral speed of 10.2 m/s and a power density of 0.45 kW/l of milling space at 80° C. In the course of this time 50 parts of water added so that the suspension remains stirrable. The grinding media again separated from the millbase by sieving, the suspension is filtered, and the presscake is washed with water and dried.

This gives 28 parts of pigment preparation.

### EXAMPLE 2 (COMPARATIVE EXAMPLE)

A pigment preparation is prepared according to EP-A-1 130 062, example 3, with the sole difference that milling takes place at 80° C.

Tests

The pigment preparation prepared according to example 1 is very strongly colored in the HS varnish, and the metallic finish is strongly colored and bright. The millbase rheology of the dispersion is evaluated as being 5 and the millbase exhibits no pseudoplasticity. The viscosity of the varnish is 1.6 sec.

The pigment preparation prepared according to example 2 shows a poorer millbase rheology, which is evaluated only as being 4, and displays an unwanted thixotropy. The viscosity of varnish is greatly increased and is 4.6 sec. The finish is very matt; the gloss measurement of the cast varnish gives a value of 21 instead of 76 in the case of example 1.

The pigment preparation prepared according to example 1 is more transparent and significantly strongly colored in the HS varnish than a commercially customary P.V.29 pigment, and the metallic finish is also substantially stronger in color.

### EXAMPLE 3

Example 1 was carried out with the sole difference that, instead of the 8.2 parts of presscake of the pigment dispersant derived from P.V.23, 1.6 parts of the pigment dispersant of the formula (III) in which V is a bivalent radical >NR⁴, R⁴ is methyl, W is a bivalent radical >NR⁵Y⁻X⁺, R⁵ is ethylene and Y⁻X⁺ is the radical —SO₃H, prepared according to EP-A-486 531 example 3, are used.

### EXAMPLE 4

Example 1 was carried out with the sole difference that, instead of the 8.2 parts of presscake of the pigment dispersant derived from P.V.23, 0.8 part of the pigment dispersant of the formula (Ill) in which V and W are a bivalent radical —NR⁵Y⁻X⁺, with R⁵ as ethylene and Y⁻X⁺ as the radical —COOH, prepared according to U.S. Pat. No. 6,413,309 example 32a, and 5.6 parts of a 28.5% presscake for the pigment dispersant of the formula (Va) 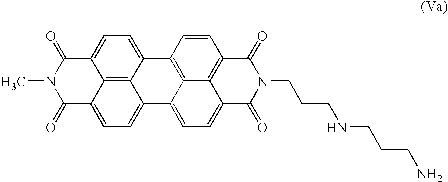
prepared according to U.S. Pat. No. 6,221,150 example 2, is used.

### EXAMPLE 5

Example 1 was carried out with the sole difference that, instead of the 8.2 parts of presscake of the pigment dispersant derived from P.V.23, 11.2 parts of 28.5% presscake of the pigment dispersant of the formula (Va) from example 4 are used.

### TESTS OF EXAMPLES 3, 4 AND 5

All three pigment preparations from examples 3, 4 and 5 give strongly colored and transparent finishes in the HS varnish, and the metallic finishes are strongly colored and bright.

1) A process for preparing a transparent perylimide pigment preparation based on at least one perylene-3,4,9,10-tetracarboxylic diimide of the formula (XXX) 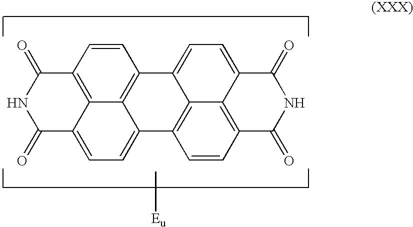
wherein
u is a number from 0 to 8 and, if u>0,
E is a chlorine or bromine atom and, where u>1, optionally a combination thereof, comprising the steps of wet-grinding a crude perylimide pigment of the formula (XXX) in a liquid, aqueous medium, in the presence of at least one pigment dispersant selected from the group consisting of perylene dispersants and dispersants derived from P.V.23, in a stirred ballmill operated at a power density of not more than 1.0 kW per liter of milling space, under the action of grinding media having a diameter of less than or equal to 5 mm and at a temperature of at least 50° C., and isolating the resulting pigment preparation.

2) The process of claim 1, wherein the perylene dispersant is a compound of the formula (II) 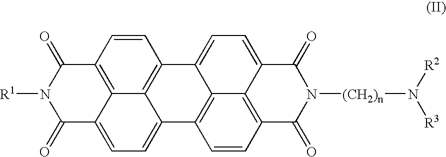
wherein
R¹ is a hydrogen atom, a hydroxyl group, amino group or a C₁-C₈-alkyl group optionally substituted by from 1 to 4 chlorine or bromine atoms or by a phenyl, cyano, hydroxyl, carbamoyl, C₂-C₄-acyl or C₁-C₄-alkoxy group or is perfluorinated or partly fluorinated;
R² and R³ independently of one another are a hydrogen atom, a substituted, unsubstituted, partly fluorinated or perfluorinated alkyl group of 1 to 20 carbon atoms, or a substituted, unsubstituted, partly fluorinated or perfluorinated alkenyl group of 2 to 20 carbon atoms;
5- to 7-membered or R² and R³ together with the nitrogen atom form a saturated, unsaturated or aromatic heterocyclic ring optionally containing a further nitrogen, oxygen or sulfur atom in the ring; and
n is a number from 1 to 6.

3) The process of claim 2, wherein R¹ is hydrogen, hydroxyethylene, methyl or ethyl, R² and R³ are each methyl or ethyl, or wherein R² and R³ together with the adjacent nitrogen atom form an imidazolyl radical or morpholinyl radical and n is the number 3.

4) The process of claim 1, wherein the perylene dispersant is a compound of the formula (III) 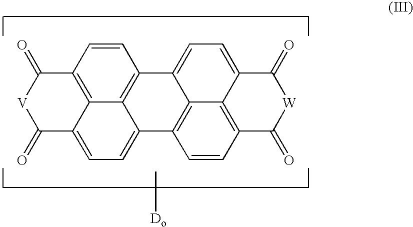
wherein
V is a bivalent radical —O—, >NR⁴ or >NR⁵—Y⁻X⁺,
W is the bivalent radical >NR⁵—Y⁻X⁺,
o is a number from 0 to 8 and, if o>0,
D is a chlorine or bromine atom and, where o>1, optionally a combination thereof,
R⁴ is a hydrogen atom, a C₁-C₁₈-alkyl group or a phenyl group unsubstituted or substituted one or more times by halogen, C₁-C₄-alkyl, C₁-C₄-alkoxy or phenylazo,
R⁵ is a C₁-C₁₈-alkylene group which within the C-C chain is optionally interrupted one or more times by a bridging link from the series —O—, —NR⁶—, —S—, phenylene, —CO—, —SO₂—, —CR⁷R⁶— or a combination thereof and wherein the radicals R⁶, R⁷ and R⁸ independently of one another are each a hydrogen atom or a C₁-C₄-alkyl group unsubstituted or substituted by a heterocyclic radical;
or R⁵ is a phenylene group unsubstituted or substituted one or more times by C₁-C₄-alkyl or C₁-C₄-alkoxy,
Y⁻ is one of the anionic radicals —SO₃⁻ or —COO⁻, and
X⁺ has the definition H⁺ or the equivalent
$$\frac{{M}^{m +}}{m}$$
of a metal cation from main groups 1 to 5 or transition groups 1, 2, or 4 to 8 of the Periodic Table of the Elements, m being 1, 2 or 3,
or M^{m+} defines an ammonium ion N⁺R⁹R¹⁰R¹¹R¹², where the substituents R⁹, R¹⁰, R¹¹ and R¹² independently of one another are each a hydrogen atom or a group selected from the group consisting of C₁-C₃₀-alkyl, C₂-C₃₀-alkenyl, C₅-C₃₀-cycloalkyl, phenyl C₁-C₈-alkylated phenyl, (C₁-C₄)-alkylene-phenyl, and a (poly)alkylenoxy group 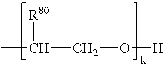
in which k is a number from 1 to 30,
R⁸⁰ is hydrogen, C₁-C₄-alkyl or, if k is >1, a combination thereof;
wherein the C₁-C₃₀-alkyl, C₂-C₃₀-alkenyl, C₅-C₃₀-cycloalkyl, phenyl or C₁-C₈-alkylated phenyl is optionally substituted by amino, hydroxyl carboxyl or a combination thereof;
or where the substituents R⁹ and R¹⁰ together with the quaternary nitrogen atom optionally form a five- to seven-membered saturated ring system optionally containing further heteroatoms selected from the group consisting of O, S and N;
or wherein R⁹, R¹⁰ and R¹¹ together with the quaternary nitrogen atom optionally form a five- to seven-membered aromatic ring system optionally containing further heteroatoms selected from the group consisting of O, S and N and to which, optionally, additional rings are fused on.

5) The process of claim 1, wherein the perylene dispersant is a compound of formula (IV) 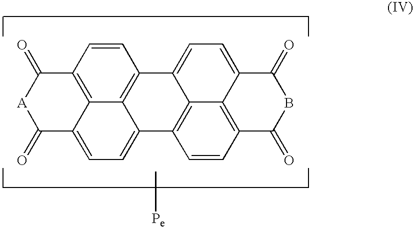
wherein
A is a cationic bivalent radical of the formula N—R¹³—NH⁺R¹⁴R¹⁵,
B is an anionic bivalent radical of the formula 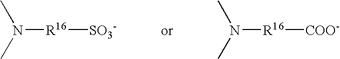
e is a number from 0 to 8, and, if e is >0,
P is a chlorine or bromine atom and, where e is >1, optionally a combination thereof,
R¹³ is a C₁-C₁₂-alkylene group, a (C₆-C₁)-aryl-(C₁-C₆)-alkylene group or a (C₆-C₁₀)-arylene group,
R¹⁴ and R¹⁵ are identical or different and are a hydrogen atom, C₁-C₂₀-alkyl or C₂-C₂₀-alkenyl, or
R¹⁴ and R¹⁵ together with the adjacent nitrogen atom form a heterocyclic ring system optional containing further heteroatom ring members selected from the group consisting of N, S and O and to which, optionally, additional rings are fused on, and
R¹⁶ is a straight-chain or branched C₁-C₁₂-alkylene group.

6) The process of claim 5, wherein
e is 0,
R¹³ is an ethylene or propylene group,
R¹⁴ and R¹⁵ are different or, identical and are an ethyl, propyl or butyl radical, or
R¹⁴ and R¹⁵ together with the adjacent nitrogen atom form a pyrrolidine, piperazine, piperidine, pyrrole, imidazoline, imidazole or morpholine ring, or form an indole, indoline or benzimidazole ring system optionally substituted by methyl,
ethyl or chlorine; and
R¹⁶ is an ethylene or propylene group.

7) The process of claim 1, wherein the perylene dispersant is a compound of formula (V) 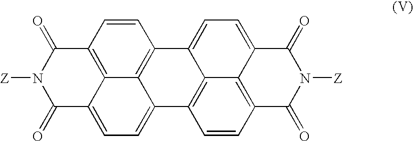
wherein the two radicals Z are identical or different and Z has the definition
Z¹, Z³ or Z⁴, with the proviso that both radicals Z are not simultaneously Z⁴, where Z¹ is a radical of the formula (Va)
—[X—Y]_{q}—[X¹—Y¹]ᵣ—[X²—NH]ₛH (Va)
wherein
X, X¹ and X² are identical or different and are a branched or unbranched C₂-C₆-alkylene radical or a C₅-C₇-cycloalkylene radical optionally substituted by from 1 to 4 C₁-C₄-alkyl radicals, hydroxyl radicals, hydroxyalkyl radicals of 1 to 4 carbon atoms, or 1 or 2 further C₅-C₇-cycloalkyl radicals;
Y and Y¹ are identical or different and are an NH—, —O—, N(C₁-C₆-alkyl) group, or 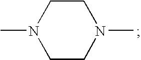
q is a number from 1 to 6;
r and s independently of one another are a number from 0 to 6;
Z³ is a radical of the formula (Vc) 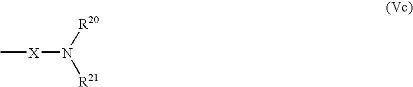
wherein
R²⁰ and R²¹ independently of one another are a hydrogen atom, a substituted or unsubstituted or partly fluorinated or perfluorinated alkyl group of 1 to 20 carbon atoms, or a substituted or unsubstituted or partly fluorinated or perfluorinated alkenyl group of 2 to 20 carbon atoms, or,
R²⁰ and R²¹ together with the nitrogen atom form a saturated, unsaturated or aromatic heterocyclic 5- to 7-membered ring optionally containing a further nitrogen, oxygen or sulfur atom in the ring; and
x is as defined above;
and wherein
Z⁴ is hydrogen, hydroxyl, amino, phenyl or C₁-C₂₀-alkyl wherein the phenyl the C₁-C₂₀-alkyl group is, optionally, substituted by one or more substituents selected from the group consisting of Cl, Br, CN, OH, C₆H₅, carbamoyl, C₂-C₄-acyl and C₁-C₄-alkoxy, and wherein the phenyl ring may also be is, optionally, substituted by NR²⁰R²¹, R²⁰ and R²¹ being as defined above, or, optionally, the C₁-C₂₀-alkyl group is perfluorinated or partly fluorinated.

8) The process of claim 1, wherein the dispersant derived from P.V.23 is a compound of the formula (I) 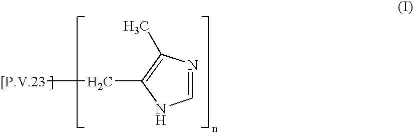
wherein
n is a number from 1 to 4.

9) The process of claim 1, wherein the pigment dispersant is used in an amount of 0.1% to 25% by weight, based on the crude perylimide pigment.

10) The process of claim 1, wherein the aqueous medium contains 0 to 10% by weight of an organic solvent, based on the water.

11) The process of claim 1, wherein the pH of the aqueous medium is greater than or equal to 9.

12) The process of claim 1, wherein the wet-grinding duration is between 10 minutes and 5 hours.

13) The process of claim 1, wherein the wet-grinding is carried out at a temperature between 60 and 95° C.

14) The process of claim 1, wherein the alkyl group of 1 to 20 atoms or the alkenyl group of 2 to 20 carbon atoms of at least one of R² and R³ is substituted by a group selected from the group consisting of hydroxyl, phenyl, cyano, chlorine, bromine, C₂-C₄-acyl and C₁-C₄-alkoxy.

15) The process of claim 4, wherein the (C₁-C₄)-alkylene-phenyl is benzyl.

16) The process of claim 6, wherein R¹⁴ and R¹⁵ are identical and are an ethyl, propyl or butyl radical.

17) The process of claim 6, wherein r and s are not both zero.

18) The process of claim 6, wherein the alkyl group of 1 to 20 atoms or the alkenyl group of 2 to 20 carbon atoms of at least one of R²⁰ and R²¹ is substituted by a group selected from the group consisting of hydroxyl, phenyl, cyano, chlorine, bromine, C₂-C₄-acyl and C₁-C₄-alkoxy.

19) A transparent perylimide pigment preparation made in accordance with the process of claim 1.

20) An automotive finish pigmented with at least one transparent perylimide pigment preparation of claim 19.

21) A high molecular mass natural or synthetic organic material pigmented with at least one transparent perylimide pigment preparation of claim 19.

22) A composition pigmented with at least one transparent perylimide pigment preparation of claim 19.

23) The composition of claim 22, wherein the composition is selected from group consisting of stoving enamels, electrophotographic toners, electrophotographic developers, magnetic toners, liquid toners, polymerization toners, specialty toners, toner binders, powders, powder coating materials, inks, and color filters.
